(19) United States
(12) Patent Application Publication
(10) Pub. No.: US 2026/0306706 A1
(43) Pub. Date: Oct. 8, 2026

(54) SELF-HEALING HYDROGEL

(71) Applicants: UCL Business Ltd, Greater London (GB); Indian Institute of Technology Indore, Indore (IN)

(72) Inventors: Deepak KALASKAR, Greater London (GB); Apurba Kumar DAS, Indore (IN)

(21) Appl. No.: 18/996,627

(22) PCT Filed: Jul. 19, 2023
(86) PCT No.: PCT/GB2023/051891

§ 371 Date: Jan. 17, 2025

(30) Foreign Application Priority Data
Jul. 20, 2022 (GB) 2210605.8

(51) Int. Cl. A61L 27/20; A61K 9/06; A61K 47/24; A61K 47/36; A61K 47/42; A61L 27/22; A61L 27/50; A61L 27/52

(52) U.S. Cl. CPC A61L 27/20; A61K 9/06; A61K 47/24; A61K 47/36; A61K 47/42; A61L 27/222; A61L 27/50; A61L 27/52; A61L 2430/02; A61L 2430/06; A61L 2430/10; A61L 2430/20; A61L 2430/34

(57) ABSTRACT

A hydrogel comprising guar gum, a (hetero)aryl diboronic acid and an aqueous medium, wherein the guar gum is crosslinked by the (hetero)aryl diboronic acid.

### FIELD OF THE INVENTION

The present invention relates to a hydrogel that is particularly suitable for 3D printing, especially in medical applications. The hydrogel may be used as an article, such as an implant, a tissue model and/or a drug delivery vehicle.

### BACKGROUND OF THE INVENTION

The lack of suitable donors for tissue or organ transplantation prolongs the suffering of many patients and can lead to death. Transplantation of a tissue or organ from one patient to another is also highly risky due to the chance of rejection and side-effects of immunosuppressant drugs. Transplantation also produces long-term expenses for health systems.

Three-dimensional (3D) bioprinting can be used to generate tissues for implantation. These tissues can be made using the cells of the patient that is to receive the tissue implant. This leads to diminished chances of rejection compared to transplantation and reduces the requirement for immunosuppressant drugs.

3D bioprinting can also be used to provide in vitro 3D disease and/or tissue models with live human cells or tissues to reduce use of animals.

The materials used to produce engineered/artificial live tissue using 3D printing are called bioinks. Bioinks can encapsulate living cells, bioactive components and/or growth factors during 3D-bioprinting.

The tissues producible by bioprinting also find application in the field of tissue models. In this regard, it is important to move away from the safety and efficacy testing of compositions in animal models for moral reasons. Indeed, the use of animal testing in the cosmetics industry has been banned. Therefore, the provision of tissue mimics for modelling disease is highly desirable for research and development in a variety of industries, including the cosmetics and pharmaceutical industries.

Furthermore, there is a desire to provide alternative vehicles for administering active ingredients for therapy.

An example of a widely used bioink is calcium alginate. However, calcium alginate-based bioinks have variations between batches; reproducibility is poor. Calcium alginate-based bioinks also suffer from using buffer containing calcium ions to achieve gelation, which can impact cellular functions and impact on the activity of active agents present in the bioink.

### SUMMARY OF THE INVENTION

According to a first aspect, the present invention provides a hydrogel comprising guar gum, a (hetero)aryl diboronic acid and an aqueous medium, wherein the guar gum is cross-linked by the (hetero)aryl diboronic acid. According to a second aspect, the present invention provides a composition comprising the hydrogel of the first aspect and an active agent. According to a third aspect, the present invention provides a pharmaceutical composition comprising the hydrogel of the first aspect or the composition of the second aspect.

It has surprisingly been found that the hydrogel of the claimed invention can be particularly suitable for forming into pre-defined 3D shapes, especially by 3D printing, especially where the (hetero)aryl diboronic acid is 1,4-benzenediboronic acid.

Furthermore, the concentration of the hydrogel can be altered to adjust its properties, in particular its viscosity. A higher concentration makes it more viscous. Thus, by adjusting the concentration of the hydrogel it can be used for different applications, such as 3D printing or injection.

The combination of guar gum with a (hetero)aryl diboronic acid, e.g. 1,4-benzenediboronic acid, alters the mechanical properties of the guar gum and allows the hydrogel to rapidly self-heal.

As a result of the combination of guar gum with a (hetero)aryl diboronic acid, the hydrogel of the present invention includes dynamic boronate ester crosslinks, which are thought to be responsible for its ability to self-heal.

It has been determined that hydrogels of the present invention can be used to enhance the mechanical properties of hydrogels made of proteins such as gelatine. Gelatine, for example, forms a very weak hydrogel that often degrades at high temperatures. However, a composite hydrogel comprising gelatine in addition to the materials required in the hydrogel of the present invention can have the benefits of gelatine hydrogels and enhanced mechanical properties.

It has been found that hydrogels of the present invention form immediately upon 3D printing. Therefore, there is insignificant delay required before subsequent layers can be printed.

In addition, there is no requirement to perform additional steps to cure, and bind together, the layers of the hydrogel, for example by the use of temperature, photo-polymerisation, pH changes, or additional crosslinking agents, such as acrylamide, which can be toxic.

The hydrogel can also display shear thinning (thixotropic) behaviour, wherein the viscosity of hydrogel decreases with increasing shear rate. This indicates good injectable and thixotropic nature of the hydrogel.

Thus, the specific components of the claimed hydrogel make it particularly suitable in the manufacture of articles, such as by 3D printing, and tissue engineering applications.

According to a fourth aspect, the present invention provides a method of manufacturing an article. The method comprises providing a hydrogel of the first aspect or a composition of the second or third aspects; and forming the hydrogel or pharmaceutical composition into a three-dimensional (3D) shape.

According to a fifth aspect, the present invention provides an article comprising a hydrogel of the first aspect and/or a composition of the second or third aspects. The article may be an implant, a tissue scaffold, a disease and/or tissue model and/or a drug delivery vehicle.

In a preferred embodiment the article has been prepared by the method of the fourth aspect, especially by 3D printing.

Hydrogels formed of polysaccharides and monoboronic acids are not suitable for 3D printing because they do not provide suitable mechanical properties or the ability to self-heal rapidly enough to retain their 3D structure after printing under substantially neutral conditions.

Importantly, using other boronic acid-containing compounds, instead of the (hetero)aryl diboronic acid required by the present invention, does not achieve the structural stability in a short period of time, that is necessary for accurate 3D printing.

Further, borax-cross-linked guar gum hydrogels have been reported (Sci. Rep. 2016, 6, No. 36497; ACS Biomater. Sci. Eng. 2018, 4, 3397-3404). However, such hydrogels are very brittle, have low flexibility, slow self-healing effect, and poor low temperature tolerance.

However, the hydrogel of the present invention has surprisingly been found to have a storage modulus of around 10 times greater than comparable guar gum borate hydrogels.

By using a (hetero)aryl diboronic acid in combination with guar gum, the claimed hydrogel includes chemically functionalised guar gum, containing boronate ester groups, with double-bonded crosslinks both between different strands of guar gum and within strands of guar gum. This provides both improved mechanical stability, and the ability to maintain the dynamic nature of the hydrogel, which makes the claimed hydrogel particularly suitable for the development of a bioink.

US 2010/0099586 A1 discusses fracturing fluid compositions and methods of fracturing subterranean formations using polyboronic compounds as crosslinking agents. It discusses that the compositions can be used to increase the viscosity of fracturing fluids. Specifically, the compositions and methods allow for lower polymer loadings because higher fracturing fluid viscosities can be achieved using less polymer than in traditional crosslinked systems.

US 2010/0099586 A1 discusses very low concentrations of the guar gum of up to 25 ppt (0.3%) in the fracturing fluids. The amount of polyboronic compound in the fracturing fluids is also very low, from around 0.02 vol % to about 0.5 vol %.

US 2010/0099586 A1 does not describe, or provide any prompting to suggest that the compositions could be modified to prepare, a hydrogel.

US 2010/0099586 A1 does not provide any prompting to explore the benefits that can be attained at higher concentrations of guar gum and/or (hetero)aryl diboronic acids. There was no discussion of 3D printing, medical injection, biopharmaceutical applications (e.g. controlled release drug delivery), self-healing, or biocompatibility in US 2010/0099586 A1.

However, the claimed invention provides a hydrogel. The claimed invention can use higher amounts of the crosslinked composition (e.g. 3% w/v) and/or use specific ratios of guar gum to (hetero)aryl diboronic acid (e.g. 8:1).

The hydrogels of the present invention have been used in to prepare highly stable articles, especially by 3D printing.

Further, the hydrogels of the present invention can be reliably and reproducibly be manufactured at scale. The materials used to prepare the hydrogels of the present invention can be fully synthetic and free of products of animal origin. Thus, GLP and GMP-compliant hydrogels of the present invention can readily be prepared.

The hydrogels of the present invention can contain low concentrations of, or no, calcium ions, therefore reducing the impact on cellular functions and on the activity of active agents present in the bioink that is felt with other hydrogels, and also increasing biocompatibility of the claimed hydrogels.

The hydrogels of the present invention have been found to be around 7.4, making the hydrogel biocompatible, and suitable for culturing living cells.

In addition, the claimed hydrogel and pharmaceutical composition can also be used as a drug delivery vehicle for the administration of one or more active agents, such as medicaments, vitamins or proteins, to a subject in need thereof. The claimed hydrogel and pharmaceutical composition may provide for controlled release of the active agent(s), for example over an extended period of time or regulated by the pH of the hydrogel (e.g. exposing the hydrogel to a more acidic environment).

Furthermore, and significantly, the hydrogels of the present invention have been shown to support gradual cellular growth during long term cell culture.

In particular, the hydrogel has been used to support human fibroblast cells.

By contrast, hydrogels of the prior art can lack biocompatibility. For example, Lu, X. et al., Colloids and Surfaces A: Physicochemical and Engineering Aspects, volume 561, 20 Jan. 2019, pages 325-331 describes self-healable hydrogels containing hydroxypropyl guar gum, acrylamide and 3-acrylamidophenylboronic acid. This document describes the use of acrylamide and 3-acrylamidophenylboronic acid to form copolymers that can cross-link the hydroxypropyl guar gum. However, acrylamide is carcinogenic and neurotoxic, and therefore not suitable for use in bioinks.

Further, the present inventors have also determined that the concentration of the guar gum and (hetero)aryl diboronic acid components of the claimed hydrogel can be modified to control the viscosity and rate of degradation of the hydrogel. As discussed in more detail by the Examples, hydrogels have been controlled to remain stable for around 50 days. Thus, the durability of the hydrogel can be modified to suit the desired application.

The claimed hydrogel may be made from a hydrogel precursor. Thus, according to a sixth aspect, the present invention provides a solid precursor of a hydrogel, wherein the precursor comprises guar gum crosslinked by a (hetero)aryl diboronic acid. Thus, the guar gum and (hetero)aryl diboronic acid may be present in a solid unit. In other words, one solid unit may contain both guar gum and the (hetero)aryl diboronic acid. The precursor may include a plurality of solid units. The solid unit may, for example, be a particle of a powder. The hydrogel of the first aspect may be made from the precursor of the sixth aspect.

### BRIEF DESCRIPTION OF THE DRAWINGS

Embodiments of the present invention will now be described by way of example with reference to the accompanying drawings, in which:

FIG. 1 shows a diagram illustrating the cross-linking of guar gum (GG) by 1,4-benzenediboronic acid (BDBA) to form a guar gum-1,4-benzenediboronic acid (GGBDBA) hydrogel;

FIG. 2 shows photographs of the GGBDBA hydrogel containing phosphate buffer;

FIG. 3 shows: a) a SEM micrograph of GGBDBA hydrogel, and b) a TEM micrograph of GGBDBA hydrogel;

FIG. 4 shows graphs illustrating the rheological performance of various concentrations of GGBDBA hydrogels;

FIG. 5 shows: a) a photograph of GGBDBA hydrogel being extruded from a needle, b) a photograph of 2 wt % GGBDBA hydrogel in a lattice pattern, and c) a photograph of 4 wt % GGBDBA hydrogel in a lattice pattern;

FIG. 6 shows CAD images of articles, and the resulting 3D-printed articles formed of GGBDBA hydrogel;

FIG. 7 shows photographs of a test to show the self-healing nature of a GGBDBA hydrogel;

FIG. 8 shows a graph illustrating human fibroblast cell count over time in the presence of a GGBDBA hydrogel;

FIG. 9 shows: a) a graph showing the absorbance of crystal violet by a GGBDBA hydrogel over time, and b) an image showing crystal violet being absorbed by the GGBDBA hydrogel;

FIG. 10 illustrates the encapsulation and release of medicaments using GGBDBA hydrogel;

FIG. 11 shows photographs illustrating the degradation of GGBDBA hydrogels in DMEM (low glucose) media over up to 50 days;

FIG. 12 shows photographs illustrating the degradation of GGBDBA hydrogels in media over 21 days;

FIG. 13 shows a photograph of a composite hydrogel made of GGBDBA and gelatine; and

FIG. 14 shows graphs illustrating the rheological performance of various concentrations of composite hydrogels made of GGBDBA and gelatine.

### DETAILED DESCRIPTION OF THE INVENTION

The present invention provides self-healing hydrogels. These can repair their structure and function following damage, similar to the healing of tissue in an organism. The hydrogels can also display good shear-thinning behaviour. Self-healing hydrogels with good shear-thinning behaviour are particularly useful in tissue engineering, 3D bio-printing and drug delivery applications.

The self-healing characteristic of the hydrogel is postulated to be due to the electrostatic attractions within the hydrogel, which can influence the hydrogel's ability to spontaneously create new bonds across the damaged interface. This, in turn, is dependent upon both the guar gum and the (hetero)aryl diboronic acid as required by the claimed invention. Hence, hydrogels comprising dynamic chemical bonds and transient physical bonds typically exhibit self-healing properties.

### Guar Gum

Guar gum (also known as guaran) is edible, low cost and an easily available polysaccharide. It is non-animal based, making it suitable for certain cultural requirements. Guar gum is a gel-forming galactomannan, obtained by grinding the endosperm of Cyamopsis tetragonolobus, a leguminous plant grown for centuries mainly in India and Pakistan. Guar is an important crop that has long been used as food for humans and animals.

Guar is a plant mainly cultivated in certain tropical regions of the world. The guar beans are edible and the extracted gum from them has a range of uses in industry such as: in textiles, where it is used to thicken dyes to allow sharp printing patterns in carpets and textile printing; in food and beverage, where it is used as a thickening, stabilizing, suspending, and binding agent, and as a fibre source; in pharmaceuticals, where it is used as a laxative, and for treating diarrhoea, irritable bowel syndrome, obesity, and diabetes; for reducing cholesterol; and for preventing atherosclerosis; in paper; in oil; and in cosmetics, where it is used in cream, lotion and cosmetics as emulsifier, stabilizer and thickener. Guar gum is cheap, readily available, edible and biologically compatible.

It will be understood that guar gum is a polysaccharide composed of galactose and mannose. The backbone is a linear chain of β 1,4-linked mannose residues to which galactose residues are 1,6-linked at approximately every second mannose (although the exact substitution is random), forming short side-branches.

However, it has been recognised that it is difficult to control the viscosity or hydration of natural guar gum, and solutions of guar gum can be instable when in solution for extended periods of time. Thus, chemical modification of guar gum is required and overcomes these problems, whilst also providing the specific benefits discussed in relation to the hydrogel itself when guar gum is combined with an aryl diboronic acid.

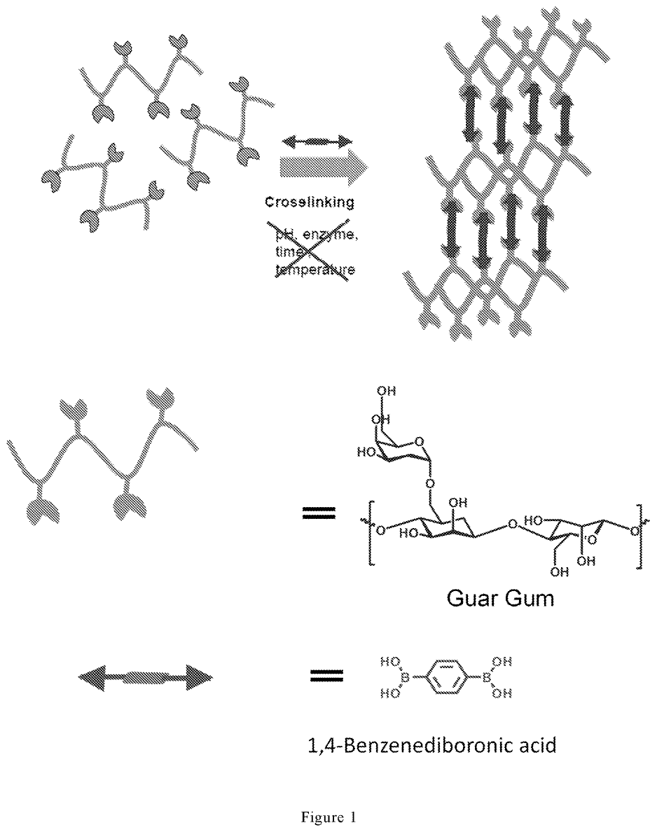

Mannose and galactose residues contain cis-1,2-diols. It is postulated that these groups can bind with boric acids (e.g. the aryl diboronic acid required by the claimed invention) to form boronate esters. This is shown by FIG. 1 of the accompanying drawings.

The aryl diboronic acids used by the present invention are therefore postulated to cross-link between the polysaccharide chains of guar gum.

The ability to form such cross-links with diboronic acids is thought to be a significant feature of guar gum that differentiates the specific use of guar gum with a diboronic acid from other polysaccharides that do not contain cis-1,2-diols. This allows the hydrogels of the present invention to have the beneficial characteristics discussed herein.

Further, solutions of guar gum in water are thought to have a higher viscosity than any other natural polysaccharide.

It is postulated that the beneficial properties of the claimed hydrogel are a result of this specific structure, and functionality, of guar gum compared to other polysaccharides.

The viscosity and hydration of the claimed hydrogel has not been difficult to control, and the stability of the hydrogel has not been a problem.

### (Hetero)Aryl Diboronic Acid

The hydrogel and the precursor include a (hetero)aryl diboronic acid. It will be understood that the term “(hetero)aryl” encompasses aryl and heteroaryl groups. It will be understood that the term “diboronic acid” means that the (hetero)aryl group includes two boronic acid groups bonded to the (hetero)aryl group.

The (hetero)aryl diboronic acid may include from 4 to 20 carbon atoms, such as from 4 to 16 carbon atoms, or from 4 to 10 carbon atoms. Preferably the (hetero)aryl diboronic acid includes from 4 to 8 carbon atoms, for example from 4 to 6 carbon atoms, more preferably from 5 to 8 carbon atoms, for example 5 or 6 carbon atoms. Most preferably the (hetero)aryl diboronic acid includes 6 carbon atoms.

Preferably the (hetero)aryl diboronic acid is an aryl diboronic acid. The term “aryl” as used herein refers to carbocyclic (i.e. not heteroatom containing) aromatic groups including phenyl, naphthyl, indenyl, and tetrahydronaphthyl groups. Aryl groups can be monocyclic or polycyclic (e.g. bicyclic), as long as at least one ring is aromatic. Aryl groups can be a five membered or six membered monocyclic ring or a bicyclic structure formed from fused five and six membered rings or two fused six membered rings. The aryl diboronic acid may include 6 or 10 carbon atoms.

The term “heteroaryl” as used herein refers to aromatic ring systems that contain heteroatoms (e.g. N, O, S) within the ring structure. Heteroaryl groups can be monocyclic or polycyclic (e.g. bicyclic), as long as at least one ring is aromatic. Heteroaryl groups can be a five membered or six membered monocyclic ring or a bicyclic structure formed from fused five and six membered rings or two fused six membered rings. Each ring may contain up to five heteroatoms typically selected from N, S and O. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom.

Examples of heteroaryl groups include pyrrole, furan, thiophene, imidazole, furazan, oxazole, oxadiazole, oxatriazole, isoxazole, thiazole, thiadiazole, isothiazole, pyrazole, triazole and tetrazole, pyridine, pyrazine, pyridazine, pyrimidine and triazine groups.

Examples of monocyclic groups are groups containing 4, 5, 6, 7 and 8 ring members, more usually 4 to 7, and preferably 5, 6 or 7 ring members, more preferably 5 or 6 ring members. Examples of bicyclic groups are those containing 8, 9 and 10 ring members.

The (hetero)aryl diboronic acid may be substituted by one or more group selected from the list consisting of: cyano, halogen (e.g. F, Cl, Br and I), N₃, —C(O)R^{z}, —C(O)OR^{z}, —OC(O)R^{z}, —C(O)NHR^{z}, —NHC(O)R^{z}, —NHC(O)NHR^{z}, —NHC(O)OR^{z}, —OC(O)NHR^{z}, —OP(O)₂OR^{z}, —S(O)₂NHR^{z}, —NHS(O)₂R^{z}, —NR^{z}₂, —NHR^{z} and —OR^{z}; wherein each R^{z} independently represents H, C1-4 alkyl or C2-4 alkenyl. For example, the (hetero)aryl diboronic acid may be substituted by one or more group selected from the list consisting of: cyano, halogen (e.g. F, Cl and Br), N₃, —C(O)R^{z}, —C(O)OR^{z}, —OC(O)R^{z}, —C(O)NHR^{z}, —NHC(O)R^{z}, —NHC(O)NHR^{z}, —NHC(O)OR^{z}, —OC(O)NHR^{z}, and —OR^{z}; wherein each R^{z} independently represents H, C1-4 alkyl or C2-4 alkenyl. Preferably the (hetero)aryl diboronic acid is not substituted with any such groups.

Preferably the (hetero)aryl diboronic acid is formed of a 6-membered (hetero)aryl ring, for example wherein the (hetero)aryl group is benzene (i.e. phenyl), pyridine, pyrazine, pyrimidine, pyridazine, or triazine. More preferably the (hetero)aryl diboronic acid is a benzenediboronic acid, most preferably 1,4-benzenediboronic acid:

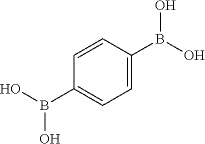 [SMILES: OB(O)c1ccc(B(O)O)cc1]

### Hydrogel Precursor

The claimed invention defines a solid precursor to a hydrogel, the precursor comprising both guar gum and a (hetero)aryl diboronic acid in a solid unit.

The hydrogel may be prepared via the precursor. The precursor may facilitate the production of the hydrogel.

The precursor may be prepared by a method comprising the steps of:
providing a solution of guar gum and a (hetero)aryl diboronic acid in an organic solvent;
adding an alkaline agent to the solution to form an alkaline solution; and
removing the organic solvent to provide the precursor.

In the precursor and/or the hydrogel, the guar gum and (hetero)aryl diboronic acid may be used in any suitable ratio. The precise ratio of guar gum to (hetero)aryl diboronic acid may be controlled to prepare a hydrogel having particular characteristics.

For example, the ratio of guar gum to (hetero)aryl diboronic acid, by weight, may be from 1:100 to 100:1, such as from 1:100 to 50:1, or from 1:100 to 20:1. The ratio of guar gum to (hetero)aryl diboronic acid, by weight, may be from 1:50 to 100:1, or from 1:20 to 100:1. Preferably the ratio by weight of guar gum to (hetero)aryl diboronic acid is from 1:10 to 100:1, for example from 1:5 to 100:1, or from 1:2 to 100:1, such as from 1:1 to 100:1.

More preferably there is an equal or greater amount of guar gum than (hetero)aryl diboronic acid by weight. For example, the ratio of guar gum to (hetero)aryl diboronic acid, by weight, may be from 1:1 to 100:1, such as from 2:1 to 100:1 (e.g. 2:1 to 40:1, or 2:1 to 20:1), or from 4:1 to 100:1 (e.g. 4:1 to 40:1, or 4:1 to 20:1). Preferably the ratio of guar gum to (hetero)aryl diboronic acid, by weight, is greater than 5:1, for example 6:1 or more, or 7:1 or more, more preferably from 6:1 to 100:1, such as 7:1 to 100:1, or 8:1 to 100:1. The ratio of guar gum to (hetero)aryl diboronic acid, by weight, may be from 6:1 to 60:1, such as from 6:1 to 40:1, or from 6:1 to 20:1, such as from 6:1 to 15:1, or from 6:1 to 10:1. The ratio of guar gum to (hetero)aryl diboronic acid, by weight, may be from 8:1 to 10:1.

The organic solvent may be water-soluble. The organic solvent may be a protic solvent. The organic solvent may be an alcohol-containing solvent, such as methanol, ethanol, propanol, or butanol. The organic solvent may be provided in an amount of from 0.1 mL/g to 200 mL/g, for example from 0.1 mL/g to 100 mL/g, or from 0.1 mL/g to 50 mL/g, relative to the total combined mass of guar gum and (hetero)aryl diboronic acid. The organic solvent may be provided in an amount of from 0.1 mL/g to 200 mL/g, for example from 1 mL/g to 100 mL/g, or from 10 mL/g to 100 mL/g, relative to the total combined mass of guar gum and (hetero)aryl diboronic acid.

Any suitable alkaline agent may be used to adjust the pH of the solution. The alkaline agent may an aqueous solution of an alkali metal or alkali earth metal hydroxide, sulfate, carbonate or phosphate. In a preferred embodiment the alkaline agent is an aqueous solution of an alkali metal or an alkali earth metal hydroxide. In a particularly preferred embodiment, the alkaline agent is an aqueous solution of sodium hydroxide, potassium hydroxide, rubidium hydroxide, caesium hydroxide, calcium hydroxide or strontium hydroxide; e.g. it may be an aqueous solution of sodium hydroxide or potassium hydroxide. In the most preferred embodiment, the alkaline agent is an aqueous solution of sodium hydroxide. The concentration of the alkaline agent may be from 0.001N to 19N, such as from 0.01N to ION, such as from 0.01N to 5N, or from 0.02N to 4N, for example from 0.05N to 2N, preferably from 0.1N to 1N, for example from 0.2N to 0.8N.

The alkaline solution may have a pH of from 7.5 to 14, such as from 8 to 13. Preferably the alkaline composition has a pH of from 10 to 12, most preferably 11. Preferably this pH is reached due to the addition of the alkaline agent. The alkaline agent may be added to the solution in any amount suitable to make the pH of the solution become alkaline, or one of the options discussed above. The pH may be determined by any method for determining pH, such as using universal indicator paper, litmus (e.g. litmus paper), or a pH meter.

The alkaline solution may be heated before the solvents are removed. Preferably the alkaline solution may be heated at a temperature of 50° C. or more, for example 60° C. or more, or 70° C. or more, for example from 50° C. to 100° C., or from 70° C. to 85° C. The heating step may be undertaken for a time period of from 30 minutes to 20 days, for example from 1 hour to 10 days, or from 1 hour to 6 days, such as from 1 hour to 4 days. The time period is preferably from 2 hours to 10 days, for example from 6 hours to 6 days, or from 12 hours to 6 days, for example from 24 hours to 4 days. Preferably the heating step is performed at a temperature of 50° C. or more, for example from 50° C. to 100° C., and for a time period of from 30 minutes to 20 days, for example from 6 hours to 6 days.

The solvents may be removed in vacuo, or by evaporation at atmospheric pressure, for example. Lyophilisation or desiccation may be used to remove any residues of water present in the precursor.

The precursor may contain 10 wt % of the organic solvent or less, such as 5 wt % or less, or 2 wt % or less. Preferably the precursor contains 1 wt % or less of the organic solvent, such as 0.5 wt % or less, or 0.1 wt % or less. The precursor may contain a trace of the organic solvent, such as 0.0001 wt % or more, such as from 0.0001 wt % to 10 wt %, or from 0.0001 wt % to 1 wt %.

The term “solid”, in this context, refers to the phase of matter of the precursor. The precursor may be in any solid form, for example as a powder, granules, flakes, clumps, or lumps. Preferably the precursor is a powder.

The precursor comprises both guar gum and a (hetero)aryl diboronic acid in a solid unit. A solid unit may be a particle of a powder or a flake, or a granule. It will be understood that this differs from a simple mixture of the guar gum and the (hetero)aryl diboronic acid, where each solid unit would include either the guar gum or the (hetero)aryl diboronic acid, but not both of these components.

In the precursor, the guar gum may be cross-linked by the (hetero)aryl diboronic acid.

The hydrogel precursor may include an active agent, such as those discussed below, and/or may be a precursor of the (pharmaceutical) composition.

### Hydrogel

The hydrogel may be made from the hydrogel precursor. For example, the hydrogel may be made by a method including the steps of:
providing the hydrogel precursor as discussed above, and
contacting the hydrogel precursor with an aqueous medium.

Optionally the hydrogel precursor is mixed with the aqueous medium.

The precursor may be added to the aqueous medium, or the aqueous medium may be added to the precursor. The precursor may be suspended and/or dispersed in the aqueous medium.

Preferably the precursor is mixed (e.g. stirred, shaken, and/or subjected to (ultra)sonication) with the aqueous medium.

Once the precursor is contacted, e.g. mixed, with the aqueous medium, the hydrogel usually forms almost instantly.

Organogels, which include organic solvents, are entirely different from the hydrogels of the present invention. Furthermore, organogels have no application in biomedical applications due to their toxicity.

The aqueous medium comprises water. The aqueous medium may be water, such as deionised and/or distilled water. The skilled person will understand that solutions with high concentrations of salt can be used to precipitate proteins, and that such solutions should not be used as the aqueous medium. As such, the aqueous medium may include 60% or more water by weight, such as 70% or more, or 80% or more. Preferably the aqueous medium includes 90% or more water by weight, such as 95% or more, or 98% or more, or 99% or more. The aqueous medium is preferably substantially free, or free, of calcium ions. For example, the aqueous medium may have a calcium ion concentration of 10 mg/L or less, such as 1 mg/L or less, or 0.1 mg/L or less. The aqueous medium (before contact with the precursor) is preferably substantially free, or free, of any organic solvent. By substantially free, it is meant that the solvent may have a concentration of the species in question of 0.05M or less, such as 0.01M or less, such as 5 mM or less, or 1 mM or less, such as 0.5 mM or less, or 0.1 mM or less. Substantially free may refer to a concentration of a species being 0.05 mM or less, such as 0.01 mM or less, or 0.005 mM or less, such as 0.001 mM or less, or 0.01 μM or less.

The aqueous medium may have a pH of from 4 to 10, such as from 5 to 9, preferably from 6 to 8, such as from 7 to 8. The pH of the aqueous medium may be particularly suitable for biocompatibility.

The precursor may suitably be contacted with any amount of the aqueous medium, such as 10% or more by weight, relative to the weight of the precursor, such as 50% or more, or 100% or more. Preferably, 200% or more by weight, relative to the weight of the precursor, of the aqueous medium is contacted with the precursor, such as 350% or more, or 500% or more. More preferably, 750% or more by weight, relative to the weight of the precursor, of the aqueous medium is contacted with the precursor, such as 1000% or more, or 1500% or more, for example 1800%. The amount of the aqueous medium that is contacted with the precursor may be 100000% or less by weight, relative to the weight of the precursor, such as 50000% or less, or 10000% or less. The amount of the aqueous medium that is contacted with the precursor may be from 10% to 100000% by weight, relative to the weight of the precursor, such as from 400% to 50000%.

The hydrogel may be separated from excess aqueous medium. Separation of the hydrogel from the aqueous medium may be performed using techniques well known to the skilled person, such as filtration or centrifugation.

The hydrogel may be described as a paste-like substance; an elastic solid and/or as a gel.

It will be appreciated that the relative amounts (ratios) of guar gum to (hetero)aryl diboronic acid in the precursor (described above) apply equally to the hydrogel itself.

The hydrogel may contain the guar gum and (hetero)aryl diboronic acid in any suitable amount. The present inventors have determined that the concentration of the guar gum and (hetero)aryl diboronic acid components of the claimed hydrogel can be modified to control the viscosity and rate of degradation of the hydrogel. The hydrogel may generally contain the combination of guar gum and (hetero)aryl diboronic acid (dry) in an amount of 0.1% or more by weight, or 0.2% or more, such as 0.4% or more, preferably 0.5% or more, or 0.7% or more, or 0.8% or more, such as 1.0% or more, or 1.5% or more, or 2.0% or more, or 2.5% or more, and/or 50% or less, such as 20% or less, preferably 10% or less, for example 8% or less, or 6% or less by weight. The hydrogel may generally contain the combination of guar gum and (hetero)aryl diboronic acid (dry) in an amount of from 0.1% to 50% by weight, such as from 0.5% to 10%.

A hydrogel containing the guar gum and (hetero)aryl diboronic acid at a concentration of below 1.5 wt %, such as 1 wt % or less has a relatively low viscosity that allows for free-flowing liquid behaviour. Such hydrogels may have particular use as injectable materials and/or drug delivery vehicles. However, concentrations of 1.5 wt % and above, such as 2 wt % and above, provide a more viscous/solid hydrogel that is not free flowing. Such hydrogels may have particular use as bioinks, for example as tissue (e.g. disease) models and/or implants. Thus, the viscosity of the hydrogel can be modified to suit the desired application.

Further, a hydrogel containing the guar gum and (hetero)aryl diboronic acid at a concentration of 6 wt % remains stable for around 50 days. However, reducing the concentration to 4 wt % makes the hydrogel remain stable for around 12 days. Thus, the durability of the hydrogel can be modified to suit the desired application.

The hydrogel may contain the combination of guar gum and (hetero)aryl diboronic acid (dry) in an amount of 0.1% or more, such as 0.3% or more, or 0.6% or more by weight. Preferably hydrogel contains the combination of guar gum and (hetero)aryl diboronic acid (dry) in an amount of 0.8% or more by weight, or 1.0% or more, such as 1.4% or more. The hydrogel may contain this combination in an amount of 5 wt % or less by weight, such as 4 wt % or less, or 3 wt % or less, preferably 2 wt % or less by weight. For example, the hydrogel may contain the combination of guar gum and (hetero)aryl diboronic acid (dry) in an amount of from 0.1% to 5% by weight, such as from 0.6% to 3%, or from 1% to 2%.

The hydrogel may contain the combination of guar gum and (hetero)aryl diboronic acid (dry) in an amount of 2% or more by weight, such as 2.5% or more, or 3% or more, or 3.5% or more, such as 4 wt % or more by weight. The hydrogel may contain the combination of guar gum and (hetero)aryl diboronic acid (dry) in an amount of 50% or less by weight, such as 20% or less, preferably 10% or less, for example 8% or less, or 6% or less by weight. For example, the hydrogel may contain the combination of guar gum and (hetero)aryl diboronic acid (dry) in an amount of from 2% to 50% by weight, such as from 2% to 10%, or from 2% to 6% by weight.

The aqueous medium may include a buffer, such as a phosphate-based buffer. Preferably the buffer is a buffer of KH₂PO₄ and K₂HPO₄ (i.e. a potassium phosphate buffer). The concentration of the buffer (before being added to the aqueous medium) may be from 0.01M to 1.0M, or from 0.05M to 0.5M, preferably from 0.08M to 0.12M, for example about 0.1M.

The aqueous medium should be suitable for survival of any cells it is to come into contact with. It will be understood that the aqueous medium should not be toxic to such cells.

In one embodiment the hydrogel contains the combination of guar gum and (hetero)aryl diboronic acid (dry) in an amount of 0.1% or more and the ratio of guar gum to (hetero)aryl diboronic acid, by weight, is from 1:100 to 100:1. In one embodiment the hydrogel contains the combination of guar gum and (hetero)aryl diboronic acid (dry) in an amount of 0.4% or more (e.g. 0.5% or more) by weight and the ratio of guar gum to (hetero)aryl diboronic acid, by weight, is from 1:100 to 100:1. In one embodiment the hydrogel contains the combination of guar gum and (hetero)aryl diboronic acid (dry) in an amount of 0.4% or more (e.g. 0.5% or more) by weight and the ratio of guar gum to (hetero)aryl diboronic acid, by weight, is from 1:100 to 60:1, for example from 2:1 to 40:1. In one embodiment the hydrogel contains the combination of guar gum and (hetero)aryl diboronic acid (dry) in an amount of from 0.1% to 50% by weight, and the ratio of guar gum to (hetero)aryl diboronic acid, by weight, is from 1:1 to 60:1.

Preferably the (hetero)aryl diboronic acid is 1,4-benzenediboronic acid, the weight ratio of guar gum to 1,4-benzenediboronic acid is from 1:1 to 60:1, and the hydrogel contains the combination of guar gum and (hetero)aryl diboronic acid (dry) in an amount of from 0.1% to 50% by weight.

The hydrogel may be left to stand for a period of time to gel, prior to use (e.g. use in 3D printing). The period of time may be 30 seconds or more, or 2 minutes or more, or 4 minutes or more, or 6 minutes or more, or 10 minutes or more. The period of time may be 30 minutes or more, such as 1 hour or more, especially where the hydrogel contains a lower amount of the (hetero)diboronic acid. The period of time may be from 30 seconds to 48 hours, such as from 2 minutes to 6 hours.

The hydrogel may comprise a protein such as gelatine. The resulting hydrogel can be termed a composite hydrogel. The hydrogel may comprise the protein, such as gelatine, in an amount of 0.1 wt % or more, such as 0.5 wt % or more, or 1 wt % or more, or 2 wt % or more, or 3 wt % or more, such as 3.5 wt % or more, or 4 wt % or more. The hydrogel may comprise the protein in an amount of 20 wt % or less, such as 10 wt % or less, or 8 wt % or less, or 6 wt % or less, for example 5 wt % or less. The hydrogel may include gelatine in an amount of from 0.1 wt % to 20 wt %, for example from 1 wt % to 10 wt %, or from 2 wt % to 6 wt %. The hydrogel may include gelatine in an amount of 0.1 wt % or more and the combination of guar gum and (hetero)aryl diboronic acid (dry) in an amount of 0.1 wt % or more, for example the hydrogel may include gelatine in an amount of from 0.1 wt % to 20 wt % and the combination of guar gum and (hetero)aryl diboronic acid (dry) in an amount of from 0.1% to 50% by weight (preferably from 2% to 10% by weight, e.g. from 2% to 6% by weight).

### Compositions

The present invention provides a composition comprising the hydrogel and an active agent.

The present invention also provides a pharmaceutical composition comprising the hydrogel or a composition thereof.

The GGBDBA hydrogel may encapsulate an active agent, for example a drug (medicament), vitamin and/or protein. The active agent may be dispersed in, for example dissolved or suspended in, the hydrogel.

The hydrogel has wide applicability as a delivery vehicle, since the physical presence of the hydrogel around the active agent can deliver the agent to an active site or tissue, for example. Thus, the nature of the agent is not particularly limited. An active agent can readily be encapsulated within the cross-linked fibrillar network of the hydrogel.

Further, the Examples show that the concentration of guar gum and (hetero)aryl diboronic acid (dry) can be modified to control the rate of degradation of the hydrogel. Thus, the hydrogel containing such an active agent may be a modified (e.g. controlled) release vehicle.

The active agent may be selected from the group consisting of therapeutic, prophylactic, diagnostic, and nutraceutical agents consisting of compounds selected from the group consisting of small molecule active agents, proteins, polypeptides, polysaccharide, and nucleic acids. The active agent may be selected from the group consisting of antibiotics, antivirals, anti-parasitics, cytokines, growth factors, growth inhibitors, hormones, hormone antagonists, antibodies and bioactive fragments thereof, antigen and vaccine formulations, anti-inflammatories, immunomodulators, and oligonucleotide drugs, paramagnetic molecules, fluorescent compounds, magnetic molecules, and radionuclides, X-ray imaging agents, and contrast agents.

The active agent may be an anticancer drug, for example doxorubicin (widely used for breast, uterine, ovarian, lung and cervical cancer treatment), or a phosphodiesterase inhibitor, or a drug for the treatment of COPD and/or asthma, such as theophylline, or a drug for treating angina and/or hypertension, or a calcium channel blocker, such as nifedipine, or a drug for treating epilepsy, or a sodium channel blocker, such as carbamazepine, or a drug for treating diabetes, such as insulin, or a drug for the treatment of hyperparathyroidism, such as a vitamin D compound, such as calcifediol.

In one embodiment the composition is a pharmaceutical composition, i.e. the composition is pharmaceutically acceptable. The hydrogel may be non-toxic, e.g. non-carcinogenic, and/or non-neurotoxic.

Preferably the hydrogel is suitable for administration to a subject such as an animal, e.g. a mammal, or a human.

The hydrogel may be used in a method of medical treatment, the method comprising administering an agent to a subject.

The pharmaceutical composition can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible/biodegradable inserts.

In some embodiments, the composition is administered systemically, for example, by intravenous or intraperitoneal administration, in an amount effective for delivery of the compositions to targeted cells. Other possible routes include trans-dermal or oral.

The composition may be administered locally, for example by injection directly into a site to be treated. The composition may be injected or otherwise administered directly to one or more tumours. Typically, local injection causes an increased localized concentration of the compositions which is greater than that which can be achieved by systemic administration. The composition may be administered locally to the appropriate cells by using a catheter or syringe. Other means of delivering the composition locally to cells include using infusion pumps or incorporating the compositions into polymeric implants, which can affect a sustained release of the active agent to the immediate area of the implant.

The composition can be provided to a cell either directly, such as by contacting it with the cell, or indirectly, such as through the action of any biological process. For example, the composition can be formulated in a physiologically acceptable carrier or vehicle, and injected into a tissue or fluid surrounding the cell. The composition can cross the cell membrane by simple diffusion, endocytosis, or by any active or passive transport mechanism.

The selected dosage depends upon the desired therapeutic effect, on the route of administration, and on the duration of the treatment desired. Generally, dosage levels of 0.001 to 10 mg/kg of body weight daily are administered to mammals. Generally, for intravenous injection or infusion dosage may be lower than for other modes of administration.

In a preferred embodiment the composition is administered by parenteral injection. This is, in particular, because the hydrogel can display shear thinning behaviour, wherein the viscosity of hydrogel decreases with increasing shear rate. This indicates good injectable and thixotropic nature of the hydrogel.

The composition can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatine, and injectable organic esters such as ethyl oleate. The formulations may be lyophilized and redissolved/resuspended immediately before use. The formulation may be sterilized by, for example, filtration through a bacteria-retaining filter, by incorporating sterilizing agents into the compositions, by irradiating the compositions, or by heating the compositions.

The composition can be applied topically. Topical administration can include application to the lungs, nasal, oral (sublingual, buccal), vaginal, or rectal mucosa. These methods of administration can be made effective by formulating the shell with transdermal or mucosal transport elements. For transdermal delivery such elements may include chemical enhancers or physical enhancers such as electroporation or microneedle delivery.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method.

The compositions loaded with one or more active agents, to deliver the one or more active agents into cells, or to a cell's microenvironment are typically for use in treatment. The uses typically include contacting the active agent-loaded composition with one more cells. The contacting can occur in vivo or in vitro. Thus, the administration may be made to a subject.

The composition may release the active agent(s) upon a change of pH, for example by increasing or decreasing the pH of the composition. Preferably the pH is increased to release the active agent(s). The increase in pH may occur through administration of the hydrogel including the active agent(s) to the acidic conditions of the stomach, urine, and/or vagina. The pH of the environment of the hydrogel may be controlled to be 7.0 or less, such as 6.5 or less, or 6.0 or less, for example 5.0 or less, e.g. from 7.0 to 0.0 or from 6.0 to 2.0.

Implants and Disease and/or Tissue Models

The hydrogels of the present invention, and articles thereof, may be used as an implant and/or as a tissue (e.g. disease) model.

The implant or tissue (e.g. disease) model may be designed to support cell or tissue growth, for example in the development and/or repair of tissue, or to provide an analogue for natural tissue. The tissue model may simulate diseased tissue.

The hydrogel, and articles thereof, may therefore be impregnated with cells and/or tissues. Cells and tissues may be selected from the list consisting of cardiac tissue, vascular implants, skin grafts, skeletal tissues such as bone, cartilage, tendon, disc, related bone, cartilage precursor cells, and bone grafts. Cells suitable for use in the hydrogel and articles thereof of the present invention may be selected from the group consisting of embryonic stem cells, adult stem cells, osteoblastic cells, pre-osteoblastic cells, chondrocytes, nucleus pulposus cells, pre-chondrocytes, skeletal progenitor cells derived from bone, bone marrow or blood, including stem cells, and combinations thereof. The cells or tissues may be of an autologous, allogenic, or xenogenic origin relative to the recipient of an implant formed by the cell culture and tissue engineering functions of the invention. It is also understood that the term tissues, as used herein, includes connective tissues, cardiac tissue, vascular implants, skin grafts and bone grafts. The hydrogel, and articles thereof, may provide an environment for at least one of the following selected from the group consisting of storage of tissue biopsy, digestion of tissue biopsy, cell sorting, cell washing, cell concentrating, cell seeding, cell proliferation, cell differentiation, cell storage, cell transport, tissue formation, implant formation, storage of implantable tissue and transport of implantable tissue.

The tissue (e.g. disease) model may be selected from the list consisting of cardiac tissue, vascular tissue, skin tissue, and skeletal tissues such as bone, cartilage, tendon, and disc. The model may be used in a method comprising storage of tissue biopsy, digestion of tissue biopsy, cell sorting, cell washing, cell concentrating, cell seeding, cell proliferation, cell differentiation, cell storage, cell transport, tissue formation, implant formation, storage of implantable tissue and transport of implantable tissue.

The tissue (e.g. disease) model may be used in a method of determining the safety of a composition, wherein the method comprises exposing the model tissue to the composition and analysing the tissue (e.g. visually) at a time point following the exposure and/or for a period following the exposure.

The implant may be selected from the list consisting of cardiac tissue, vascular implants, skin grafts, skeletal tissues such as bone, cartilage, tendon, disc, related bone, cartilage precursor cells, and bone grafts.

### Manufacture of Articles

The hydrogel and/or pharmaceutical composition of the present invention may be used to prepare articles. The articles may include the aforementioned tissue (e.g. disease) models and implants.

It has been recognised that the hydrogel displays shear thinning properties, allowing the hydrogel to be injected and yet form a solid article. Furthermore, the hydrogel displays self-healing properties, allowing layers of the hydrogel to bond to one another without requiring additional heat and/or adhesive.

The fourth aspect of the present invention provides a method of manufacturing an article, wherein the method comprises:
providing the hydrogel of the first aspect or the composition of the second or third aspects; and
forming the hydrogel or pharmaceutical composition into a three-dimensional (3D) shape, for example by subjecting the hydrogel to 3D printing, CNC machining, casting, rotational moulding, vacuum forming, injection moulding, extrusion, and/or blow moulding to form the article.

Preferably the hydrogel is formed using 3D printing.

In one embodiment the method includes forming the 3D shape into or onto a substrate and, after forming the 3D shape, releasing the article from the substrate.

The present disclosure provides the subject-matter of the following clauses:
1. A hydrogel comprising guar gum, a (hetero)aryl diboronic acid and an aqueous medium, wherein the guar gum is cross-linked by the (hetero)aryl diboronic acid.
2. The hydrogel of clause 1, wherein the (hetero)aryl diboronic acid includes from 4 to 10 carbon atoms.
3. The hydrogel of any one of clause 1 or clause 2, wherein the (hetero)aryl diboronic acid is 1,4-benzenediboronic acid.
4. The hydrogel of any one of clauses 1 to 3, wherein the ratio of guar gum to (hetero)aryl diboronic acid is from 1:1 to 100:1 by weight.
5. The hydrogel of any one of clauses 1 to 4, wherein the hydrogel contains the combination of guar gum and (hetero)aryl diboronic acid in an amount of from 0.5% to 10% by weight.
6. The hydrogel of any one of clauses 1 to 5, wherein the aqueous medium is a phosphate-based buffer.
7. The hydrogel of any one of clauses 1 to 6, wherein the hydrogel further comprises gelatine.
8. The hydrogel of clause 7, wherein the hydrogel comprises gelatine in an amount of from 2% to 10% by weight.
9. A composition comprising the hydrogel of any one of clauses 1 to 8 and an active agent.
10. The composition of clause 9, wherein the composition is a pharmaceutical composition.
11. The composition of clause 9 or clause 10, wherein the active agent is a therapeutic, prophylactic, diagnostic, and/or nutraceutical agent comprising compounds selected from the group consisting of small molecule active agents, proteins, polypeptides, polysaccharide, and nucleic acids.
12. The composition of any one of clauses 9 to 11, wherein the active agent is selected from the group consisting of antibiotics, antivirals, anti-parasitics, cytokines, growth factors, growth inhibitors, hormones, hormone antagonists, antibodies and bioactive fragments thereof, antigen and vaccine formulations, anti-inflammatories, immunomodulators, and oligonucleotide drugs, paramagnetic molecules, fluorescent compounds, magnetic molecules, and radionuclides, X-ray imaging agents, and contrast agents.
13. A method of manufacturing an article, wherein the method comprises:
   providing a hydrogel of any one of clauses 1 to 8 or a composition of any one of clauses 9 to 12; and
   forming the hydrogel or pharmaceutical composition into a three-dimensional (3D) shape.
14. The method of clause 13, wherein the article is formed by 3D printing.
15. An article comprising a hydrogel of any one of clauses 1 to 8 or a composition of any one of clauses 9 to 12.
16. The article of clause 15, wherein the article is manufactured by the method as defined by clause 13 or clause 14.
17. The article of clause 16, wherein the article is a tissue model or an implant.
18. The article of clause 17, wherein the tissue model is selected from the list consisting of cardiac tissue, vascular tissue, skin tissue, and skeletal tissues such as bone, cartilage, tendon, and disc.
19. The article of clause 17, wherein the implant is selected from the list consisting of cardiac tissue, vascular implant, skin graft, skeletal tissue such as bone, cartilage, tendon, disc, related bone, cartilage precursor cells, and bone graft.
20. A solid precursor of a hydrogel, wherein the precursor comprises guar gum crosslinked by a (hetero)aryl diboronic acid.

### EXAMPLES

Guar gum, sodium hydroxide, 1,4-benzenediboronic acid and doxorubicin hydrochloride were purchased commercially. The sodium phosphate buffer (PB) solution (0.1 M, pH 7.46) was prepared using Milli Q water.

The percentage given in relation to any hydrogel refers to the percentage solid content (i.e. guar gum and 1,4-benzenediboronic acid) by weight, relative to the total weight of the hydrogel.

### Synthesis of GGBDBA Hydrogel Precursor

Guar gum (GG “extrapure”, purchased from Sisco Research Laboratories Pvt Ltd, 1.6 g) and 1,4-benzenediboronic acid (BDBA, 0.2 g) were mixed (weight ratio of 8:1) and dissolved in ethanol (30 mL, approximately 20 mL/g, absolute). NaOH (0.5 N) was added dropwise into the mixture until the pH of mixture become basic (pH-11). The resulting mixture was heated under reflux (80° C.) for 48 h. After 48 h, the solvents were removed under vacuum and the resulting light green residue/pale-yellow powder was dried in a desiccator (2 days) to provide dried crosslinked powder ready for hydrogel preparation. Optionally the powder was washed five times by ethanol to remove impurities. Each time, ethanol (10 mL) was poured to the solid residue and the obtained suspension was scratched and sonicated for 10-15 minutes; the resulting suspension was allowed to settle down and the supernatant was discarded. After washing, an off-white solid residue was obtained that was dried under vacuum and stored in a desiccator.

### Synthesis of GGBDBA Hydrogel

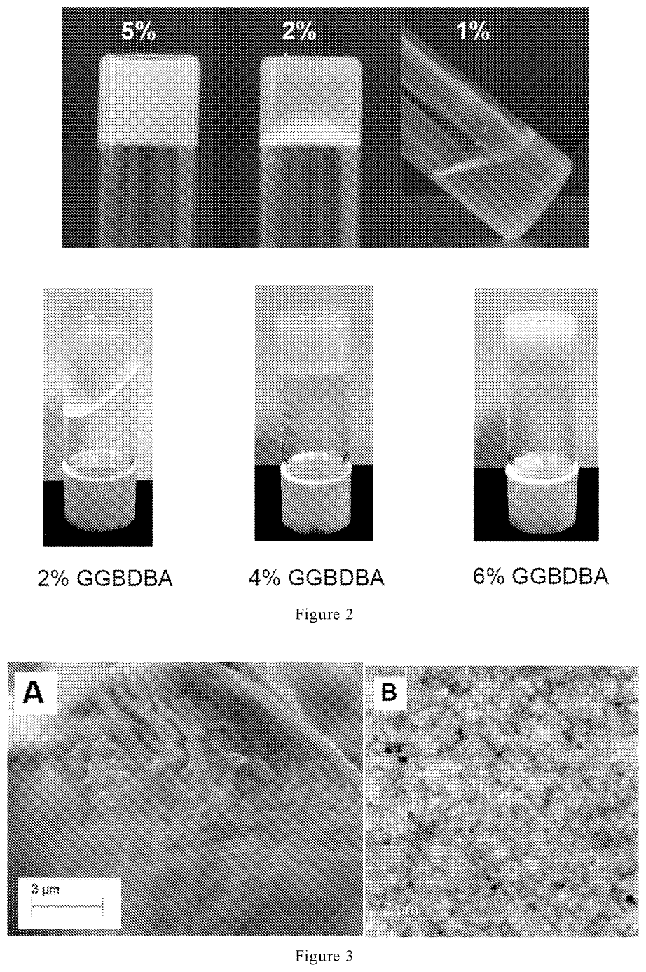

The dried crosslinked powder was used for hydrogelation. To prepare the GGBDBA hydrogel at different concentrations (1, 2, 3, 4, 5, 6% w/v), first appropriate amounts of the synthesized powder (e.g. 30 mg for 3% w/v) were taken in a 5 mL glass vial. Then desired volume of phosphate buffer (pH 7.4; 0.1 M, 1 mL for 3% w/v example) was added into the vial and the solution was sonicated for 10-15 minutes and/or mixed for 5 minutes using a vortex mixer. Instantly, the self-supported hydrogels were formed at all mentioned concentrations. The hydrogel was stored at 20° C. for 6 h before performing other experiments. FIG. 2 shows samples of the hydrogel at the different concentrations prepared. The samples are inclined or inverted to show their viscosity.

### Tetrahydroxydiboron Hydrogel (Comparative)

In a comparative study, the same procedure was followed using guar gum and another diboronic acid (tetrahydroxydiboron). However, the tetrahydroxydiboron cross-linked guar gum failed to form the hydrogel under same phosphate buffer conditions.

### Gelation Time

Hydrogels of the claimed invention were synthesised with different ratios of guar gum to 1,4-benzenediboronic acid, as shown in the table below. The gelation times of each of these hydrogels was determined by mixing the precursor with phosphate buffer in a test tube, inverting the test tube, and determining the time taken for the mixture to achieve a gel-like consistency.

The results are shown in the table below.

| GG:BDBA ratio (w/w) | Gelation time | |
|---|---|---|
| 4:1 | 72 | h |
| 5:1 | 48 | h |
| 8:1 | 4-5 | min |
| 10:1 | 4-5 | min |

It can be seen that a ratio of guar gum to diboronic acid of greater than 5:1 provides a short gelation time of 4-5 minutes. Lower ratios of guar gum provided longer gelation time, presumably due to the lower relative amount of cross-linking due to the lower amount of the diboronic acid.

The other experiments described herein were all conducted with a hydrogel containing 8:1 GG:BDBA by weight. It will be appreciated that the hydrogel is left to gel before being used for 3D printing. Once the hydrogels had formed a gel, any of the tested hydrogels were suitable for use in applications such as 3D printing.

### FT-IR Spectroscopy

The FT-IR spectrum of lyophilized GG-BDBA was recorded using a Bruker Tensor 27 spectrophotometer. These solid-state experiments were performed using the KBr pellet making technique with a scan range between 600 and 4000 cm-1 over 64 scans at a resolution of 4 cm-1 and an interval of 1 cm-1.

The FT-IR spectrum showed the formation of boronate ester groups.

### Morphological Study (Electron Microscopy)

Transmission electron microscopic (TEM) measurements were performed to evaluate the self-assembled morphology of the hydrogel. TEM images were taken by using transmission electron microscope (Model: Jeol 100 keV system) with an accelerating voltage of 300 kV. The diluted aqueous solution of the hydrogel (5% V/V) was dried on a carbon-coated copper grid (300 mesh) by slow evaporation in air and then allowed to dry separately under a reduced pressure at room temperature. Phosphotungstic acid (0.2%, w/v) was used as negative staining.

Field-emission Gun-scanning electron microscopic study was performed by using Carl Zeiss scanning electron microscope (FE-SEM Supra 55 Zeiss). The diluted aqueous gel sample was dried on a glass cover slip and coated with gold for SEM analysis with an operating voltage of 5 kV.

The SEM and TEM images are shown in FIG. 3 of the accompanying drawings.

The SEM image of GGBDBA (FIG. 3a) shows distinct flaky and stretchy surface morphology.

The TEM image of GGBDBA (FIG. 3b) displays fibrillar networks.

### Rheological Tests

Rheological analyses were performed at 25° C. using an Anton Paar Physica MCR 301 Rheometer. The viscoelastic properties of the hydrogel were measured by measuring the storage modulus (G′) and loss modulus (G″). The freshly prepared hydrogel was placed on a rheometer plate and kept hydrated by using solvent trap. A parallel plate of diameter 25 mm was used with a Trugap of 1 mm. The dynamic strain sweep experiment was performed to determine the region of deformation of hydrogel in which linear viscoelasticity is valid. The exact strains of the hydrogel were determined by linear viscoelastic regime at a constant frequency of 10 rad s⁻¹. To evaluate the mechanical strengths of the G-quadruplex hydrogel, frequency sweep experiments were performed at 0.5% strain and within frequency range of 1-600 rad s⁻¹. The change in viscosity was measured as a function of the shear rate (0.1-1000 s⁻¹) by rheometer to study the shear-thinning behaviour of the hydrogel.

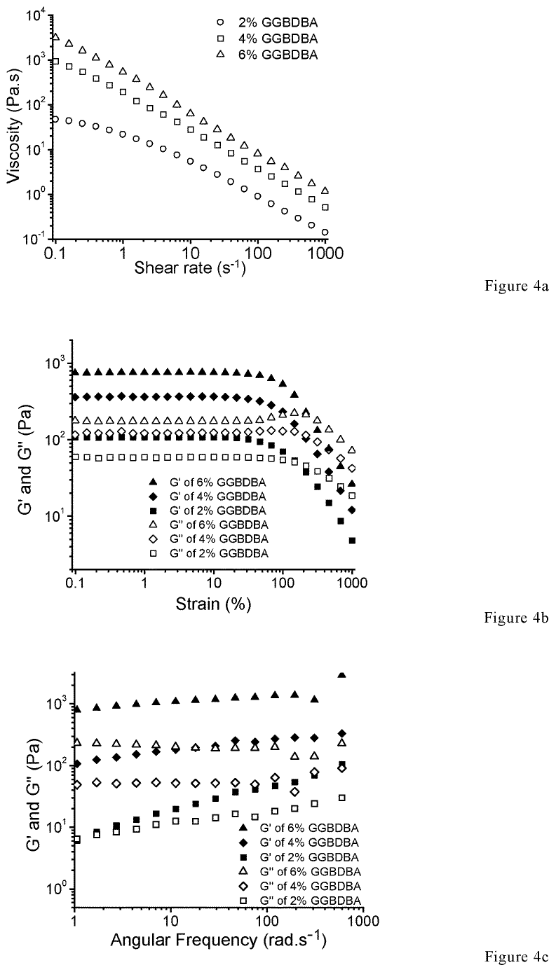

Shear thinning behaviour of the hydrogel was observed (FIG. 4a), wherein the viscosity of hydrogel decreases with increasing shear rate. This indicates good injectable and thixotropic nature of the hydrogel.

The oscillatory strain sweep experiments at constant frequency are shown in FIG. 4b. These results depict that the GGBDBA hydrogel maintained its viscoelastic property up to high strain of 200%.

The dynamic frequency sweep experiments shown in FIG. 4c depict that the storage modulus (G′) remained larger than its loss modulus (G″) as it was examined as a function of frequency, exhibiting solid-like rheology.

Further, it was found that the storage modulus of the GGBDBA hydrogel was around 10 times greater than comparable guar gum borate hydrogels (J. Agric. Food Chem. 2019, 67, 2, 746-752).

These properties are important for hydrogel applications in the form of injectable materials through syringes and extrusion-based 3D-printing and bioprinting processes, and show that the resulting article can be sufficiently stable for implantation, for example.

### Injectability

The injectability of the GGBDBA hydrogels (2, 4, and 6%) was tested by extruding the hydrogel by a syringe through a 21-G needle.

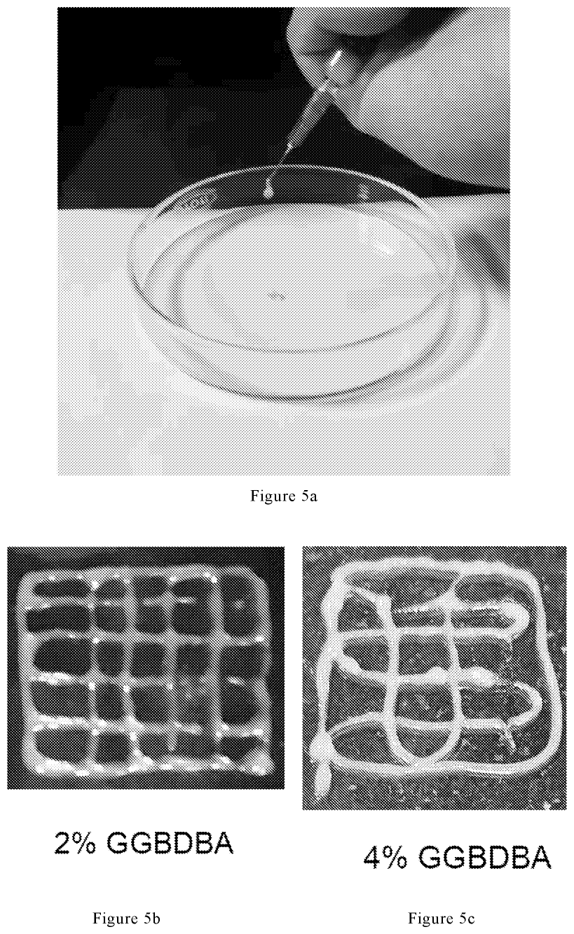

As shown in FIG. 5a, the hydrogels were successfully extruded through the needle. This illustrates the potential to use the hydrogels in 3D printing and as a vehicle for drug delivery by injection.

### Printability

The ability to use hydrogels (2, 4, and 6%) in printing was tested by extruding the hydrogel by a syringe through a 21-G needle, to form a pattern on a substrate.

As shown in FIG. 5b and FIG. 5c, the hydrogels successfully produced a pattern on the substrate. This illustrates that the hydrogels can be used to print articles.

### 3D Printing

Scaffolds were designed with Solidworks (2016, Dassault Systemes), the slicing software used was Slic3r (v. 1.2.9). Hydrogel was prepared by mixing 2% article into 0.1M, pH 7.4 sodium phosphate buffer by weight per volume. After vortexing, formed hydrogel was used directly for 3D printing using INKREDIBLE+ printer (CELLINK).

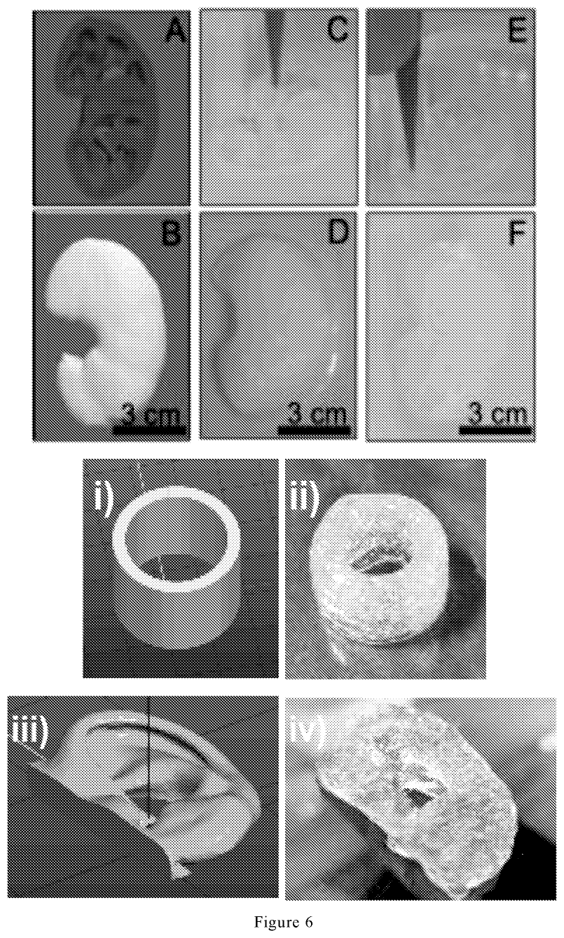

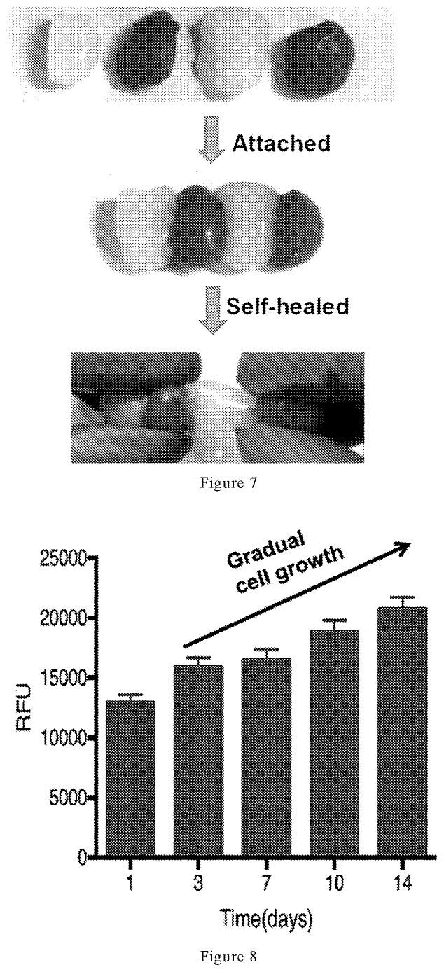

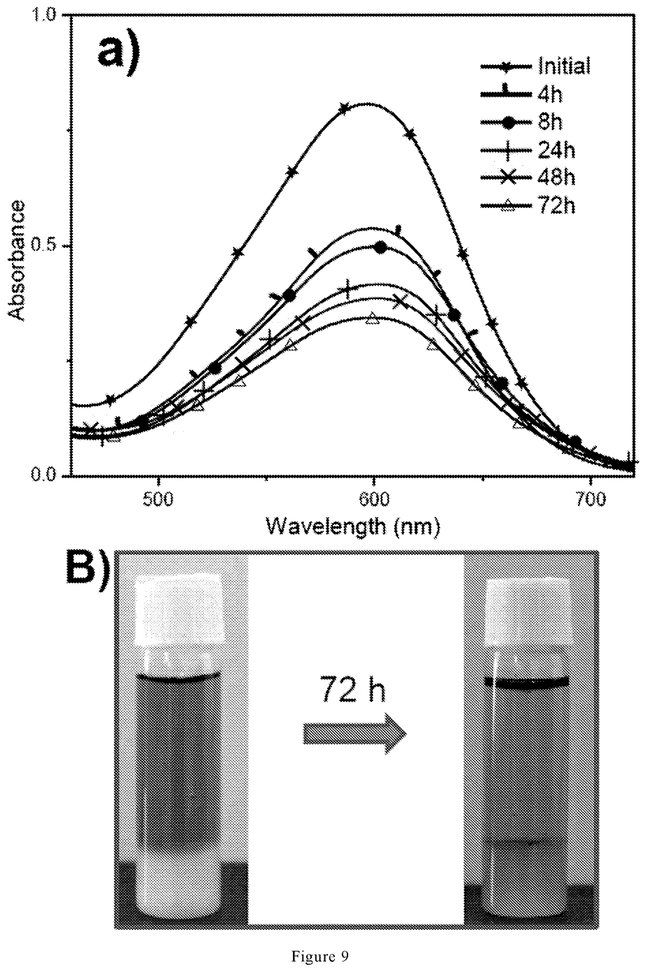

FIG. 6 illustrates the CAD designs to be printed (left-most images; A, B, i, iii) and the resulting hydrogel implants that have been made from the hydrogels (C, E (both during production), D, F, ii, iv).

This shows that the hydrogels can be used to produce articles by 3D printing.

### Self-Healing Properties

The self-healing properties of GGBDBA gel (6%) was examined by preparing two portions of the hydrogel, with a trace amount of Rhodamine B (RdB) added in one piece to give a colour difference (FIG. 7, top). The two portions of hydrogel were each split into two, to provide four portions that were brought together alternately (FIG. 7, middle).

After resting for 2-3 minutes the portions of hydrogel had self-healed. The resulting hydrogel was subjected to hanging and stretching tests. The tests confirmed that self-healing of the hydrogel had occurred. Furthermore, the tests confirmed that the boronate cross-linking between chains of guar gum is reversible and dynamic in nature. This is thought to be due to the rapid formation/hydrolysis of the boronate ester.

### Cell Compatibility

2% hydrogel, sterilised under UV light for 30 minutes, was mixed with human fibroblast cells inside a cell culture hood. The mixture was placed in a cartridge and the cartridge was sealed.

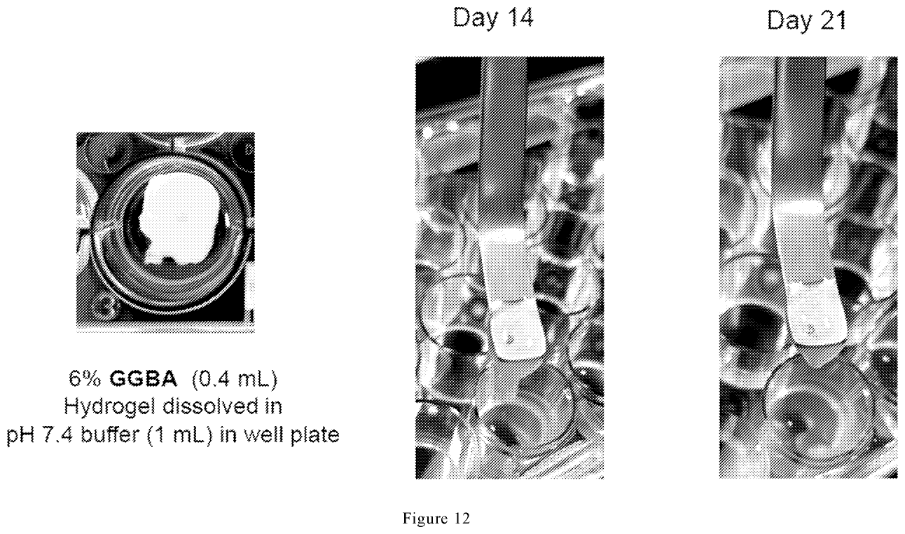

Over 14 days of cell culture in the presence of the hydrogel, gradual cell growth was observed (as illustrated by FIG. 12).

This shows that the hydrogel is non-toxic and is an effective cell growth medium.

### Nutrient Uptake

The ability of the hydrogel to absorb nutrients to support life within the hydrogel was examined by studying the absorbance of crystal violet from solution by UV-vis spectroscopy over the course of 72 hours.

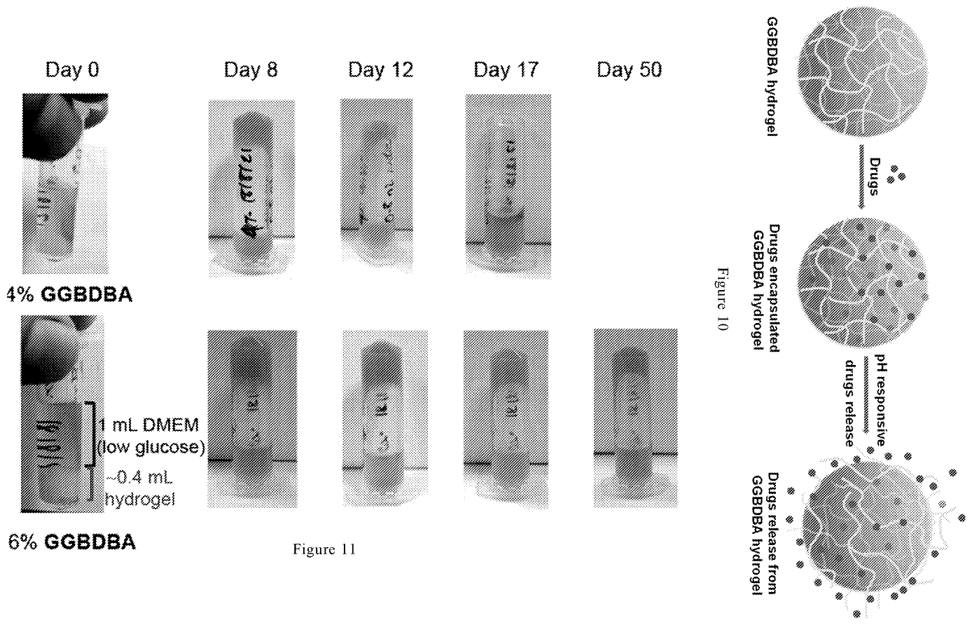

The results are shown in FIG. 9 of the accompanying drawings, wherein FIG. 9a shows the UV-vis absorbance of the solution of crystal violet and FIG. 9b shows photographs of the test vial before and after the 72-hour test period.

The results show a gradual uptake in the amount of crystal violet from the solution into the hydrogel.

Therefore, the hydrogel can allow nutrient uptake to support life within the hydrogel.

### Drug Delivery

FIG. 10 shows how the hydrogels of the present invention can be used as a delivery vehicle for an active agent. For example, a drug (i.e. therapeutic agent) can be encapsulated into the hydrogel. The drug may be released from the hydrogel and/or the hydrogel may decompose/break apart, for example in response to a change in pH.

### Controllable Degradation

Samples of the hydrogel (0.4 mL, 4 wt % or 6 wt % solid content) were contacted with DMEM (Dulbecco's Modified Eagle Medium, low glucose, 1 mL) in a vial. The samples were left for a time period (8 days, 12 days, 17 days, 50 days) before being inverted to visually determine whether the hydrogel had degraded.

The results are shown in FIG. 11 of the accompanying drawings. In particular, the 4 wt % hydrogel degraded after around 12 days, whereas the 6 wt % hydrogel degraded after around 50 days in the media.

Another sample of hydrogel (6 wt % solid content, 0.4 mL) was contacted with pH 7.4 buffer (1 mL) in a well plate. The sample was incubated in the buffer at 37° C. and the supernatant buffer was changed after each 24 hour period that passed, to simulate in vivo conditions.

FIG. 12 of the accompanying drawings shows the results of this study. After day 14 the hydrogel showed some degradation. More degradation was visible at day 21.

Therefore, the concentration of the hydrogel can be modified to control the rate of degradation of the hydrogel, and articles made therefrom.

### Enhancement of Peptide Hydrogels

Composite hydrogels containing GGBDBA and gelatin (GL) were prepared by mixing GGBDBA and GL. The concentration of GL was fixed at 4% w/v. Different concentrations of GGBDBA (0.5%, 1%, 1.5%, 2%, 3% and 4% w/v) were mixed with the GL. The mixture was incubated at 37° C. to form a gel. GL (4% w/v, without GGBDBA) was also prepared, as a control.

Hydrogels were formed at each concentration of GGBDBA. However, no hydrogel was formed with the control sample.

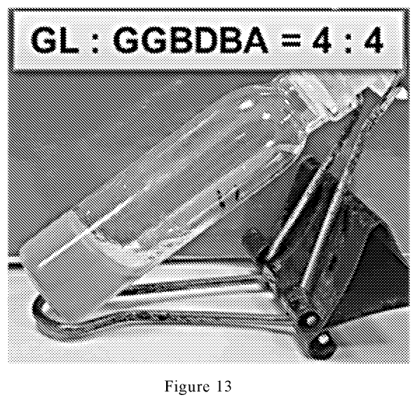

A particularly stable hydrogel was observed for the formulation of 4% GGBDBA-4% GL, which is shown by FIG. 13.

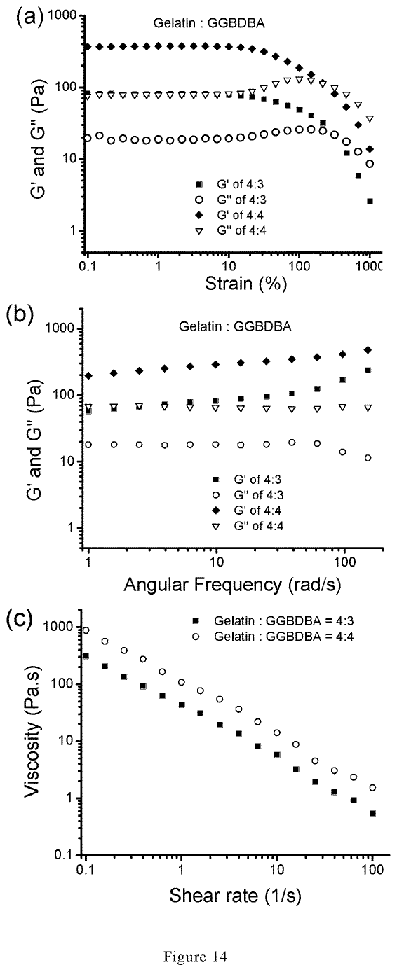

The rheological performance of the composite hydrogels 3% GGBDBA-4% GL and 4% GGBDBA-4% GL are shown by FIG. 14. FIG. 14a shows an amplitude sweep experiment to analyze LVE; FIG. 14b shows a frequency sweep experiment at constant shear strain 1% to analyze G′ and G″ of the composite hydrogel; and FIG. 14c shows a viscosity vs shear rate study.

The amplitude strain sweep experiment was performed to measure the linear viscoelastic region (LVE). The results (FIG. 14a) show that both of the tested formulations have an LVE region up to 20%. The frequency sweep experiment was employed at shear strain of 1%, to determine the viscoelastic properties of the compositions. Both formulations showed a higher storage modulus (G′) than loss modulus (G″), which represents the viscoelastic nature of the composite hydrogels. However, hydrogel consisting of 3% GGBDBA-4% GL shows G′ value of 60 Pa, whereas 4% GGBDBA-4% GL has 220 Pa. Therefore, increasing in the concentration of GGBDBA increases the viscoelastic nature of solution. Hence, G′ and G″ of the hydrogel are enhanced as the concentration of GGBDBA increased (FIG. 14b). Further, the viscosity of the 4% GGBDBA-4% GL formulation shows higher viscosity than 3% GGBDBA-4% GL. However, upon increasing the concentration of GGBDBA in 4% GL, the viscosity of the solution increased.

The composite hydrogel of GGBDBA and GL does not require the addition of a crosslinking agent, unlike some prior art composite hydrogels. Yet, the composite hydrogel can be adapted to optimise its function and viscosity.

1. A hydrogel comprising guar gum, a (hetero)aryl diboronic acid and an aqueous medium, wherein the guar gum is cross-linked by the (hetero)aryl diboronic acid.

2. The hydrogel of claim 1, wherein the hydrogel contains the combination of guar gum and (hetero)aryl diboronic acid in an amount of 0.5% or more by weight.

3. The hydrogel of claim 2, wherein the hydrogel contains the combination of guar gum and (hetero)aryl diboronic acid in an amount of 0.5% or more, by weight, and the ratio of guar gum to (hetero)aryl diboronic acid, by weight, is from 2:1 to 40:1.

4. The hydrogel of claim 2, wherein the amount of the combination of guar gum and (hetero)aryl diboronic acid is from 0.5% to 10% by weight.

5. The hydrogel of claim 1, wherein the (hetero)aryl diboronic acid is an aryl diboronic acid.

6. The hydrogel of claim 5, wherein the (hetero)aryl diboronic acid is 1,4-benzenediboronic acid.

7. The hydrogel of claim 1, wherein the aqueous medium is a phosphate-based buffer.

8. The hydrogel of claim 1, wherein the hydrogel further comprises gelatine.

9. The hydrogel of claim 8, wherein the hydrogel comprises gelatine in an amount of from 2% to 10% by weight.

10. A composition comprising the hydrogel of claim 1 and an active agent.

11. A pharmaceutical composition comprising the hydrogel of claim 1.

12. The composition of claim 10, wherein the active agent is a therapeutic, prophylactic, diagnostic, and/or nutraceutical agent comprising compounds selected from the group consisting of small molecule active agents, proteins, polypeptides, polysaccharide, and nucleic acids.

13. (canceled)

14. A method of manufacturing an article, wherein the method comprises the steps of:
providing a hydrogel of claim 1; and
forming the hydrogel into a three-dimensional (3D) shape.

15. The method of claim 14, wherein the article is formed by 3D printing.

16. An article comprising a hydrogel of claim 1.

17. The article of claim 16, wherein the article is manufactured by a method comprising the steps of:
providing a hydrogel of claim 1; and
forming the hydrogel into a three-dimensional (3D) shape.

18. The article of claim 16, wherein the article is a tissue model or an implant.

19. The article of claim 18, wherein the article is a tissue model selected from the list consisting of cardiac tissue, vascular tissue, skin tissue, and skeletal tissues, including bone, cartilage, tendon, and/or disc.

20. The article of claim 18, wherein the article is an implant selected from the list consisting of cardiac tissue, vascular implant, skin graft, and skeletal tissue, including bone, cartilage, tendon, disc, related bone, cartilage precursor cells, and/or bone graft.

21. A solid precursor of a hydrogel, wherein the precursor comprises guar gum crosslinked by a (hetero)aryl diboronic acid.
